(19) United States
(12) Patent Application Publication
(10) Pub. No.: US 2014/0206938 A1
(43) Pub. Date: Jul. 24, 2014

(54) ENDOSCOPE DEVICE

(71) Applicant: Samsung Electronics Co., Ltd., Gyeonggi-do (KR)

(72) Inventors: Kaihotsu WATARU, Gyeonggi-do (KR); Mun-Kue Park, Gyeonggi-do (KR); Jin-Won Lee, Gyeonggi-do (KR); Su-Kwang Lim, Seoul (KR)

(73) Assignee: Samsung Electronics Co., Ltd., Gyeonggi-do (KR)

(21) Appl. No.: 14/159,946

(22) Filed: Jan. 21, 2014

(30) Foreign Application Priority Data
Jan. 22, 2013 (KR) 10-2013-0007013

(51) Int. Cl. A61B 1/00; A61B 1/01

(52) U.S. Cl. CPC A61B 1/00142; A61B 1/01
USPC 600/114; 600/129

(57) ABSTRACT

An endoscope device is provided. The endoscope device includes a housing connected to a manipulator, to be inserted into a human body, a replaceable tube detachably provided in the housing, and a guide tube fixedly passing through the housing, to allow the replaceable tube to be inserted thereinto.

### PRIORITY

This application claims priority under 35 U.S.C. §119(a)to a Korean Patent Application filed on Jan. 22, 2013 in the Korean Intellectual Property Office and assigned Serial No. 10-2013-0007013, the entire disclosure of which is incorporated herein by reference.

### BACKGROUND OF THE INVENTION

1. Field of the Invention

The present invention generally relates to an endoscope device, and more particularly, to an endoscope device that provides a replaceable contamination-prone component for single use.

2. Description of the Related Art

Endoscopes are instruments that are inserted into the interior of a hollow organ or cavity of the body, such as the stomach, the large intestine, the sigmoid colon, the bronchial tubes, and the like, to examine the interior of the body and diagnose any possible disease. Along with increased interest in health care, endoscopy has recently become an essential part of a health examination. To reuse the endoscope for another examinee or patient, an inserted part of the endoscope is sterilized with a sterilization agent.

FIG. 1 illustrates an endoscope of the related art, FIG. 2 illustrates the interior of the endoscope illustrated in FIG. 1, and FIG. 3 is a simplified view of the endoscope illustrated in FIG. 2. Referring to FIGS. 1, 2 and 3, an endoscope 10 includes a channel tube 12, a suction tube 13, a Y branch 14 branched from the channel tube 12 into the suction tube 13 and the outside of the endoscope 10, and suction valves 15 that control suction, inside a housing 11 provided with a nozzle at a front end of the housing 11. The endoscope 10 may be divided into a part A inserted into the body of an examinee or patient and a part B kept outside the body of the examinee. After the endoscope 10 is used for one examinee, the endoscope 10 must be sterilized to be reused for another examinee. Specifically, as part A of the endoscope 10 is inserted into the body of the examinee, part A must be sterilized before the endoscope 10 is reused for another examinee in order to eliminate any possible contaminating material remaining due to the direct contact with the body of the examinee. Although part B of the endoscope 10 is not brought into direct contact with the body of the examinee, suctioned material may be introduced from the channel tube 12, the suction tube 13, the Y branch, or the suction valves 15 into part B. Therefore, part B must also be sterilized. However, the corners or branched portions of the channel tube 12 or the suction tube 13, the space between the suction valves 15, or the inside of the channel tube 12 or the suction tube 13 may not be fully sterilized. Even a slightly careless sterilization may lead to the accumulation of contaminating materials, which may not be eliminated even with further sterilization. Moreover, a contaminant material introduced into the gap between components of the endoscope 10 is not easy to remove with sterilization.

Furthermore, while the endoscope 10 is cleansed to prevent contamination, the provision of a camera module installed on the front end of the endoscope 10 makes high-temperature sterilization difficult. As a user must then sterilize the endoscope manually, labor costs increase while sterilization reliability and stability decrease. Due to installation of an additional cleaning valve to clean the interior of the tube in which a material moves, the endoscope 10 gets complicated in structure or the cleaning valve may act as another contamination source. If a contaminated endoscope is used for an examinee, various problems may be produced, including infection of the examinee and diagnosis errors.

Many endoscope configurations have been proposed for sterilization or disinfection of an endoscope. Techniques in the related art to prevent contamination of an endoscope are disclosed in Japanese Laid-Open Patent Application 2004-016393 and Japanese Laid-Open Patent Application 2003-284675. According to the related art, to prevent contamination of an inserted part of an endoscope, the endoscope is sheathed or draped and inserted into an intended organ of a patient, or a suction tube or a channel tube is disposed on the exterior of the endoscope and thus used just once.

In the case of the sheathed or draped endoscope, the endoscope should be covered in the sheath or drape, and it is difficult to insert the sheathed or draped endoscope into an intended position. Moreover, as a camera is also covered at the front end of the endoscope, a clear field of view is not secured, thus obstructing accurate examination of an intended body part. Since the sheath or drape covers part A of the endoscope inserted into a part to be examined, part A may be protected from contamination. However, the interior of the tube in which a material is suctioned or part B is vulnerable to contamination. Thus, the tube or part B requires additional sterilization.

In the case where a suction tube and a channel tube are disposed at the exterior of an endoscope and used just once, the outward exposure of the channel tube or the suction tube may cause pain to an examinee or make insertion difficult, when the endoscope is inserted into the body of the examinee. In addition, because the cross section of the inserted part of the endoscope does not have an “O” shape, collected tissue may be damaged.

### SUMMARY OF THE INVENTION

Aspects of the present invention are to address at least the above-mentioned problems and/or disadvantages and to provide at least the advantages described below. Accordingly, an aspect of the present invention is to provide an endoscope device that facilitates insertion of an endoscope into an intended position and reduces possible insertion-incurred pain of an examinee by maintaining the cross section of the endoscope in an ‘O’-shape and disposing a suction tube or a channel tube inside the endoscope.

Another aspect of the present invention is to provide an endoscope device that prevents overall contamination of an endoscope and increases stability of the endoscope by allowing a contamination-vulnerable component of the endoscope to be replaced with a new one after one-time use.

In accordance with an aspect of the present invention, an endoscope device is provided. The endoscope device includes a housing connected to a manipulator, the housing configured to be inserted into a human body, a replaceable tube detachably provided in the housing, and a guide tube fixedly passing through the housing, to allow the replaceable tube to be inserted thereinto.

In accordance with another aspect of the present invention, an endoscope device is provided. The endoscope device includes an inserted part to be inserted into a human body, and a non-inserted part that is not inserted into the human body. The inserted part is provided with a tube unit replaceable after one-time use and is connected to the non-inserted part.

### BRIEF DESCRIPTION OF THE DRAWINGS

The above and other aspects, features, and advantages of certain exemplary embodiments of the present invention will be more apparent from the following description taken in conjunction with the accompanying drawings, in which:

FIG. 1 illustrates an endoscope of the related art;

FIG. 2 illustrates the interior of the endoscope illustrated in FIG. 1;

FIG. 3 illustrates a simplified structure of the endoscope illustrated in FIG. 2;

FIG. 4 illustrates an endoscope device according to an embodiment of the present invention;

FIG. 5 illustrates a simplified structure of the endoscope device illustrated in FIG. 4;

FIG. 6 is a view of a front end of the endoscope device illustrated in FIG. 4;

FIG. 7 is a sectional view of the front end of the endoscope device illustrated in FIG. 4;

FIG. 8 illustrates a state where a replaceable tube is removed from a guide tube in the endoscope device illustrated in FIG. 4;

FIGS. 9A and 9B illustrate a suction valve according to an embodiment of the present invention;

FIGS. 10A and 10B are simplified views of an active state of the suction valve illustrated in FIGS. 9A and 9B; and

FIGS. 11A to 11D illustrate a process of replacing a replaceable tube, a suction tube, and a Y branch from the endoscope device according to an embodiment of the present invention.

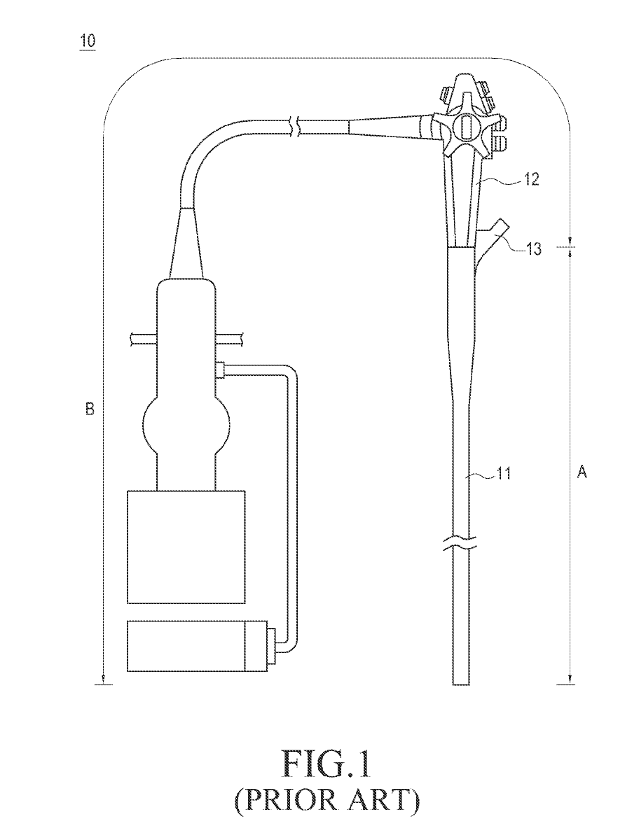

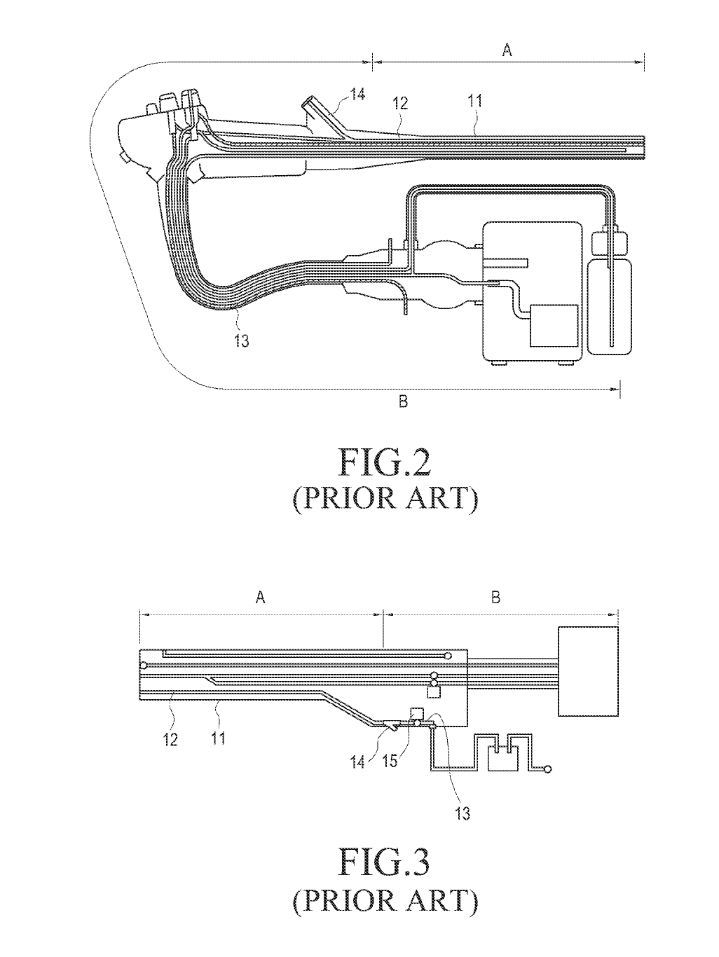

Throughout the drawings, like reference numerals will be understood to refer to like parts, components, and structures.

### DETAILED DESCRIPTION OF EMBODIMENTS OF THE PRESENT INVENTION

The following description with reference to the accompanying drawings is provided to assist in a comprehensive understanding of embodiments of the invention as defined by the claims and their equivalents. It includes various specific details to assist in that understanding but these are to be regarded as examples. Accordingly, those of ordinary skill in the art will recognize that various changes and modifications of the embodiments described herein can be made without departing from the scope and spirit of the invention. In addition, descriptions of well-known functions and constructions may be omitted for clarity and conciseness.

The terms and words used in the following description and claims are not limited to their dictionary meanings, but, are merely used to enable a clear and consistent understanding of the invention. Accordingly, it should be apparent to those skilled in the art that the following description of embodiments of the present invention is provided for illustration purposes only and not for the purpose of limiting the invention as defined by the appended claims and their equivalents.

It is to be understood that the singular forms “a,” “an,” and “the” include plural referents unless the context clearly dictates otherwise. Thus, for example, reference to “a component surface” includes reference to one or more of such surfaces.

By the term “substantially” it is meant that the recited characteristic, parameter, or value need not be achieved exactly, but that deviations or variations, including for example, tolerances, measurement error, measurement accuracy limitations and other factors known to those of skill in the art, may occur in amounts that do not preclude the effect the characteristic was intended to provide.

Embodiments of the present invention are provided to achieve the above-described technical aspects of the present invention. In an implementation, defined entities may have the same names, to which the present invention is not limited. Thus, embodiments of the present invention can be implemented with same or ready modifications in a system having a similar technical background.

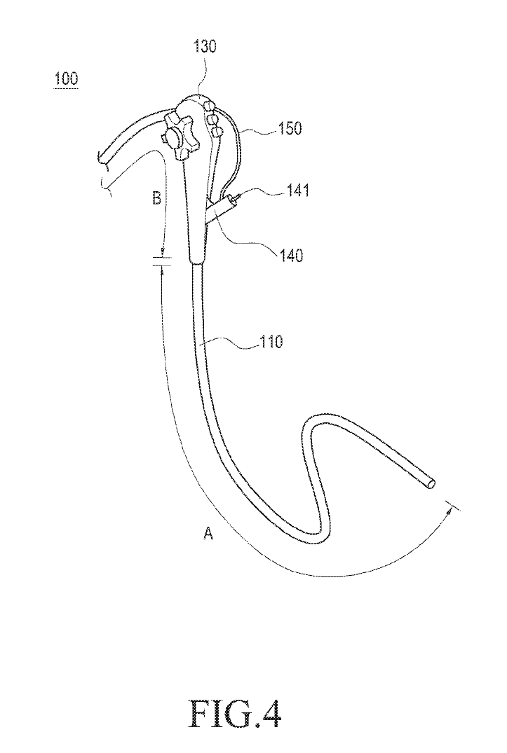

An endoscope device 100 of the present invention is intended to overcome the problem of contamination of an endoscope caused by insertion of the endoscope into a human body, or to overcome difficulty in full sterilization of the endoscope even though a part of the endoscope is not inserted. Particularly, the present invention provides endoscope device 100 that allows a tube inserted into a human body and thus directly contaminated to be replaced with a new one and that facilitates replacement or sterilization of a component that is not easy to sterilize or vulnerable to secondary contamination due to frequent use, although not directly contacting a contaminant. With reference to FIGS. 4 to 11D, the endoscope device 100 of the present invention will be described below.

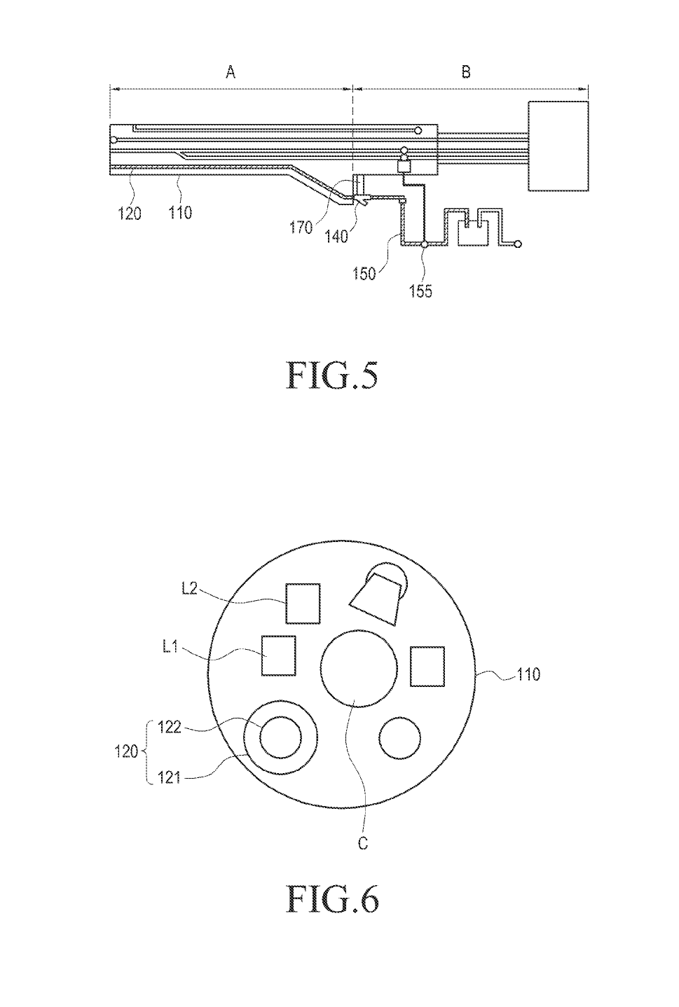

FIG. 4 illustrates an endoscope device according to an embodiment of the present invention, FIG. 5 illustrates the interior of the endoscope device illustrated in FIG. 4, and FIG. 6 illustrates a front end view of the endoscope device of FIG. 4. Referring to FIGS. 4-6, the endoscope device 100 of the present invention is divided into an inserted part A and a non-inserted part B. As is implied by its appellation, the inserted part A of the endoscope device 100 is inserted into a human body and thus brought into direct contact with the human body, to examine the interior of the body. The non-inserted body B of the endoscope device 100 is connected to the inserted part A. When an examinee or patient is observed with the endoscope device 100, the non-inserted part B is not inserted into the body. Instead, the non-inserted part B is used to control the inserted part A, specifically operates or adjusts the inserted part A, and controls the function and operation of the inserted part A.

Specifically, the inserted part A includes a tube unit 120 which is provided to take a blood sample or a tissue sample from an examinee or into which a procedure instrument is inserted. A replaceable tube 122 is provided in the tube unit 120. In the tube unit, a suction tube 150 for suction or a Y branch 140 is provided from the non-inserted part B. The suction tube 150 or the Y branch 140 is replaceable or detachable from the tube unit 120, for ready sterilization. The inserted part A includes a housing 110, the replaceable tube 122, and a guide tube 121.

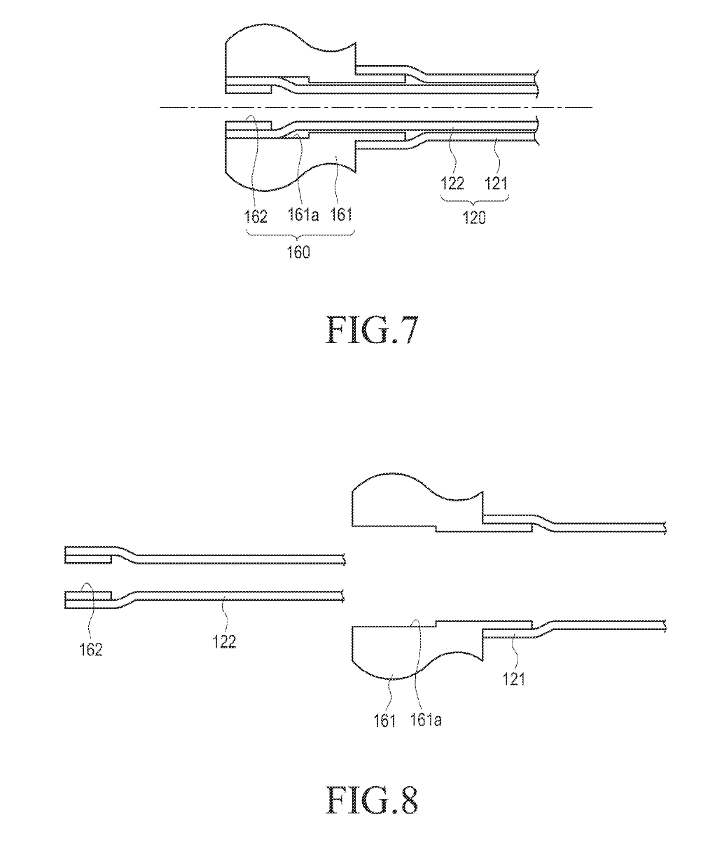

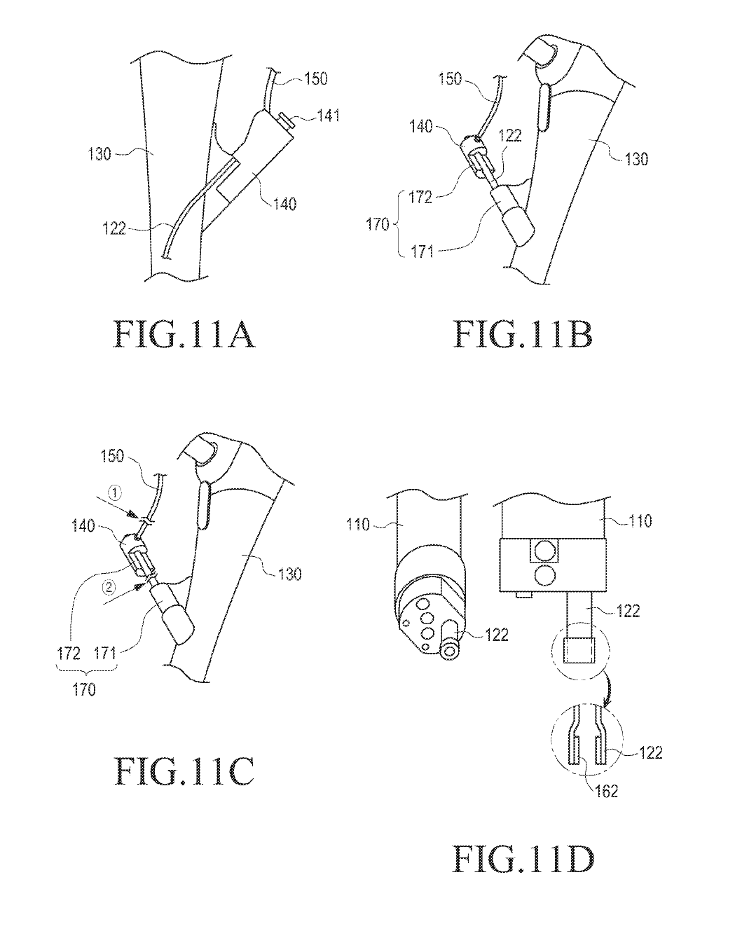

Referring to FIGS. 4 and 6, the housing 110 is connected to a manipulator 130 provided in the non-inserted part B and inserted into a human body. The housing 110 has an ‘O’-shape that facilitates insertion into the human body. A camera C or lights L1 and L2 are provided in the ‘O’-shaped housing 110 to allow an examiner or doctor to observe an intended body part of an examinee, and the tube unit 120 is provided in the ‘O’-shaped housing 110, into which instruments that perform various procedures such as blood suction or tissue collection are inserted. As seen in FIG. 7, the housing 110 is provided at its front end with a press-fitting unit 160 that fixes the replaceable tube 122 to the guide tube 121 and seals the gap between the replaceable tube 122 and the guide tube 121, thus preventing contamination of the guide tube 121. The press-fitting unit 160 will be described below in greater detail. The tube unit 120, specifically the guide tube 121 and the replaceable tube 122, pass through the housing 110 from one end to the other end. The replaceable tube 122 extends outward from the other end of the housing 110, specifically from the manipulator 130 connected to the rear end of the housing 110. A Y branch fixing surface 171, as seen in FIGS. 11B and 11C, is formed at the other end of the housing 110, specifically at the manipulator 130 in order to fix the Y branch 140.

The tube unit 120 includes the guide tube 121 and the replaceable tube 122. The guide tube 121 passes through the housing 110, in the vicinity of the installation position of the camera C or the lights L1 and L2. The guide tube 121 is firmly fixed in the housing 110. Specifically, the guide tube 121 is cylindrical, passing through the housing 110 from one end to the other end. The replaceable tube 122 is detachably mounted into the guide tube 121 so that the replaceable tube 122 may be inserted into or removed from the guide tube 121.

To enable collection of blood or tissue from an examinee, the replaceable tube 122 is hollow. When the examinee is examined by means of the endoscope device 110 with the replaceable tube 122 mounted inside the guide tube 121, the replaceable tube 122 is brought direct contact with a body part to be examined, thus being contaminated with blood or tissue. Thereafter, the replaceable tube 122 is removed from the guide tube 121 after one-time use and a sterilized new replaceable tube 122 is inserted into the guide tube 121 to examine another examinee. That is, once the replaceable tube 122 is used for one examinee, the replaceable tube 122 is removed from the endoscope device 100 and a new replaceable tube 122 is inserted into the endoscope device 100, so that another examinee may be observed with the sterilized endoscope device 100, to ensure against contamination.

FIG. 7 is a sectional view of the front end of the endoscope device illustrated in FIG. 4, and FIG. 8 illustrates a state where the replaceable tube is removed from the guide tube in the endoscope device illustrated in FIG. 4. Referring to FIGS. 7 and 8, the press-fitting unit 160 includes a distal end 161 and a sealing member 162. The distal end 161 is provided at the front end of the guide tube 121, and is fixed at the front end of the housing 110. A press-fitting recess 161 a is formed to have a step into an inner surface of the front end of the distal end 161. The press-fitting recess 161 a is formed to have a larger diameter than a rear end of the distal end 161. When the sealing member 162 is fit in the press-fitting recess 161a, the sealing member 162 brings the replaceable tube 122 into close contact with the press-fitting recess 161a, sealing the gap between the replaceable tube 122 and the guide tube 121. Therefore, the sealing member 162 prevents introduction of contaminants into the space between the replaceable tube 122 and the guide tube 121. The sealing member 162 is cylindrical and hollow, formed to a size that makes the sealing member 162 fit into the press-fitting recess 161a. The sealing member 162 includes a ferrule and thus is inserted into the press-fitting recess 161a semi-forcedly. However, the sealing member 162 is not limited to this specific configuration. As far as the sealing member 162 is configured to prevent introduction of a contaminant such as blood or tissue of an examinee into the space between the guide tube 121 and the replaceable tube 122, any configuration is available to the sealing member 162.

In the endoscope device 100 having the above-described configuration, particularly in the inserted part A, the replaceable tube 122 is inserted through the distal end 161 along the guide tube 121, protruding from the rear end of the housing 110. Thus the replaceable tube 122 is contained in the housing 110, particularly in the guide tube 121. After the replaceable tube 122 is inserted into the guide tube 121, the front end of the replaceable tube 122 is positioned at the front end of the guide tube 121, specifically in the press-fitting recess 161a of the distal end 161. When the sealing member 162 is forced-fit into the press-fitting recess 161a, the replaceable tube 122 closely contacts the inner surface of the distal end 161, specifically the inner surface of the press-fitting recess 161a, so that the gap between the guide tube 121 and the replaceable tube 122 is sealed. Therefore, when the front end of the inserted part A is inserted to examine an examinee, introduction of a contaminant into between the replaceable tube 122 and the guide tube 121 may be prevented and thus contamination inside the guide tube 121 may be prevented.

The non-inserted part B includes the manipulator 130, the suction tube 150, and the Y branch 140. The non-inserted part B further includes a device that takes or cleanses blood or tissue introduced from the inserted part A. The Y branch 140 or the suction tube 150 is mounted in the manipulator 130. An examiner controls operation of the inserted part A inserted into an organ or body cavity of an examinee by means of the manipulator 130. The manipulator 130 is provided with the Y branch fixing surface 171 to attach or remove the Y branch 140.

As seen in FIGS. 11a-11D, the Y branch 140 connects the replaceable tube 120 to the suction tube 150. The Y branch 140 is detachably installed with respect to the manipulator 130. The Y branch 140 includes a branch portion 141 other than a portion that connects the replaceable tube 120 to the suction tube 150, so that various procedure instruments may be inserted into the replaceable tube 120 from the outside of the endoscope device 100 through the branch portion 141. Referring to FIGS. 11B and 11C, a Y branch fixture 170 is provided to install the Y branch 140 to the manipulator 130 or removes the Y branch 140 from the manipulator 130. The Y branch fixture 170 includes the Y branch fixing surface 171 provided in the housing 110 and an engagement portion 172 provided in the Y branch 140. In an embodiment of the present invention, the Y branch fixing surface 171 is shaped to have a protruding semi-cylindrical shape and the engagement portion 172 is also semi-cylindrically shaped, so that the Y branch fixing surface 171 and the engagement portion 172 are engaged to form a cylindrical shape. The engagement portion 172 is engaged with or removed from the Y branch fixing surface 171 in a one-touch fashion. While the one-touch mechanism is not shown, the one-touch mechanism may be realized by forming a groove around the Y branch fixing surface 171 and forming a protrusion on the Y branch 140 to be elastically inserted into or removed from the groove, such that when the Y branch 140 is mounted on the Y branch fixing surface 171, the protrusion is caught into the groove with the replaceable tube 122 connected to the suction tube 150 through the Y branch 140. In this state, when force is applied to the Y branch 140 such as by pressing or rotation, the protrusion is released from the groove, thus removing the Y branch 140 from the housing 110. In this manner, the Y branch 140 may be mounted to the housing 110 or removed from the housing by a simple manipulation. However, the mechanism of installing or removing the Y branch 140 to or from the housing 110 is not limited to this specific mechanism. As far as it installs or removes the Y branch 140 to or from the housing 110, any mechanism is available.

Referring again to FIG. 11A, the suction tube 150 is connected to the rear end of the replaceable tube 122 through the Y branch 140. When blood or tissue is taken from an examinee at an observed position, the suction tube 150 suctions the blood or tissue and controls its suction. The suction tube 150 is exposed outward from the housing 110. Specifically, the suction tube 150 extends from one end of the Y branch 140 at an end of the housing 110. Thus the suction tube 150 is also replaceable in the housing 110. That is, the suction tube 150 may be cut from the Y branch 140 and thus replaced with another suction tube. Obviously, the Y branch 140 is also detachable from the housing 110 and thus replaceable with a new one. In an embodiment of the present invention, components vulnerable to direct and indirect contamination, specifically, the replaceable tube 122, the suction tube 150, and the Y branch 140, are detachably installed on the housing 110 and are replaceable with new ones in the endoscope device 100. Accordingly, after the endoscope device 100 is used for an examinee, the directly or indirectly contaminated components are replaced and thus the endoscope device 100 may be used continuously, protected against contamination. Particularly, the Y branch 140 may be removed from the housing 110 by cutting off the replaceable tube 122 and the suction tube 150 from the Y branch 140 and then replaced.

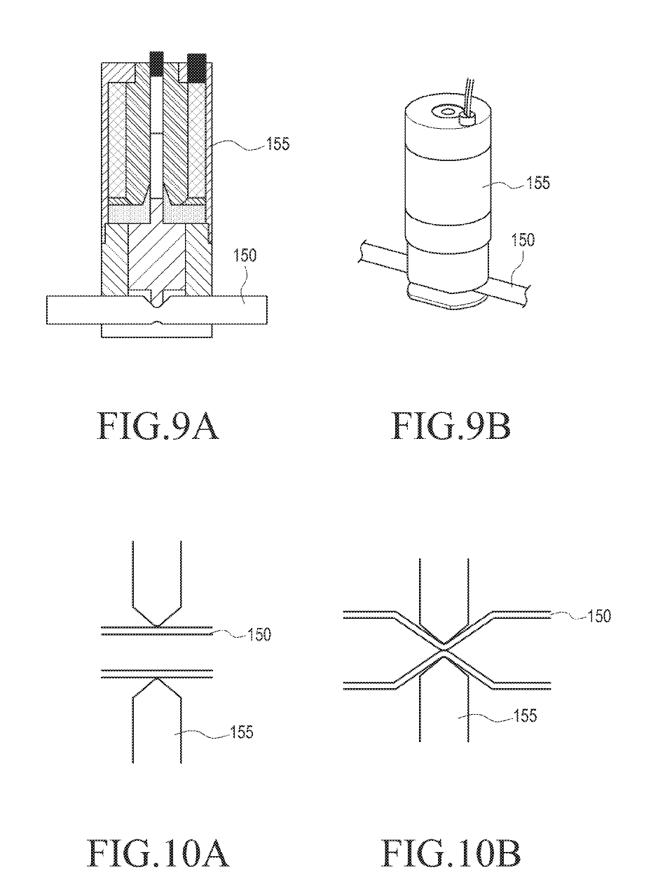

FIGS. 9A and 9B illustrate a suction valve according to an embodiment of the present invention, and FIGS. 10A and 10B are simplified views of an active state of the suction valve illustrated in FIGS. 9A and 9B.

Referring to FIGS. 9A to 10B, the suction tube 150 is provided with a suction valve 155 that controls suction of blood or tissue from an examinee. In an embodiment of the present invention, the suction value 155 is disposed at the exterior of the suction tube 150 so as to turn on/off the suction tube 150 and avoid direct contact with blood, fluid, or tissue introduced into the suction tube 150. The suction value 155 includes a solenoid valve, which is provided to pinch the outer surface of the suction tube 150 branched from the Y branch 140. For example, when a voltage is applied to the solenoid valve, the solenoid valve presses or releases the outer surface of the suction tube 150, thus turning on or off the suction tube 150. An electromagnetic valve such as the solenoid value according to the embodiment of the present invention is configured to press or release the outer surface of the suction tube 150, and thus does not contact the inner surface of the suction tube 150 at all. As a result, the suction valve 155 is not exposed to possible contamination of the inner surface of the suction tube 150, thus requiring no additional sterilization. While a solenoid valve is provided as an example of the suction valve 155 in the embodiment of the present invention, the present invention is not limited to a solenoid valve and any electromagnetic or electromechanical valve is available.

FIGS. 11A to 11D illustrate a process of replacing the replaceable tube 122, the suction tube 150, and the Y branch 140 in the endoscope device according to the embodiment of the present invention.

Referring to FIGS. 11A to 11D, the Y branch 140 is disposed at the rear end of the housing 110, specifically at a position where the replaceable tube 122 is exposed. The Y branch 140 is installed to an outer surface of the housing 110 and connects the suction tube 150 exposed outwardly from the housing 110 to the replaceable tube 122 inside the housing 110. As the engagement portion 172 of the Y branch 140 is engaged with or removed from the Y branch fixing surface 171 at the rear end of the housing 110, the Y branch 140 is removed from or attached to the manipulator 130. That is, as indicated above, the Y branch fixing surface 171 protrudes from the rear end of the housing 110. When the engagement portion 172 is engaged with the Y branch fixing surface 171, the replaceable tube 122 is contained inside the Y branch 140 and connected to the suction tube 150. Thus, the Y branch 140 covers the replaceable tube 122 protruding from the Y branch fixing surface 171, while the engagement portion 172 of the Y branch 140 and the Y branch fixing surface 171 are in an engaged state. While the Y branch 140 does not contact a human body directly, contaminants difficult to sterilize may remain in the Y branch 140, due to possible indirect contamination during endoscopy or prolonged use of the endoscope device 100. In this case, the Y branch 140 is also replaceable and thus the endoscope 100 may be used for another examinee in a sterilized state.

An operation of components that are assembled to the endoscope device 100 divided into the inserted part A and the non-inserted part B and then replaced will be described with reference to FIGS. 8 to 11D.

As stated before, the replaceable tube 122 is inserted through the distal end 161, passes through the guide tube 121, and then extends from the rear end of the housing 110 and terminates at press fitting unit 160, as seen in FIG. 11D. Then the replaceable tube 122 is connected to the suction tube 150 through the Y branch 140 provided at the manipulator 130 at the rear end of the housing 110. After the replaceable tube 122 is inserted into the guide tube 121, the sealing member 162 is forced-fit into the press-fitting recess 161a from the front end of the replaceable tube 122, thus sealing the gap between the guide tube 121 and the replaceable tube 122. When the Y branch 140 is engaged with the Y branch fixing surface 171 at the other end of the housing 110, the Y branch 140 covers the replaceable tube 122 protruding from the Y branch fixing surface 171 and the replaceable tube 122 is branched toward and then connected to the suction tube 150. Accordingly, as the replaceable tube 122, the Y branch 140, and the suction tube 150 are replaced with new ones, an examinee may be examined with the endoscope device 100 free of contamination.

Further, after the endoscope device 100 with the new replaceable tube 122, Y branch 140, and suction tube 150 is used for an examinee, the replaceable tube 122, the Y branch 140, and the suction tube 150 may be contaminated due to contact with a human body and difficult to sterilize despite non-contact with the human body. These contaminated components may be replaced with new ones to examine another examinee. Specifically, the Y branch 140 is removed from the housing 110 by applying a specific force to the Y branch fixing surface 171. Then with the replaceable tube 122 and the suction tube 150 connected to each other, the Y branch 140 is removed from the housing 110. When the suction tube 150 and the replaceable tube 122 are cut from both ends of the Y branch 140, the Y branch 140 is completely removed from the housing 110. The Y branch 140 may be replaced with a new one or may be sterilized and then reconnected. After the Y branch 140 is removed from the housing 110 by cutting the suction tube 150 and the replaceable tube 122 from both ends of the Y branch 140, the sealing member 162 forced-fit into the front end of the housing 110 is released from the press-fitting unit 160. Along with removal of the press-fitting unit 160, the replaceable tube 122 is pulled from the front end of the housing 110, specifically the front end of the guide tube 121 and thus removed from the guide tube 121. The replaceable tube 122, the suction tube 150, and the Y branch 140 removed from the housing 140 are replaced with new ones, or are sterilized and then reconnected to the housing 110. The endoscope device 100 with the new or sterilized replaceable tube 122, suction tube 150, and Y branch 140 may be used securely for another examinee.

As is apparent from the above description, the endoscope device has components vulnerable to direct or indirect contamination caused by examination of the interior of a human body, which are configured to be replaced. Therefore, a part inserted into the body or a part that is not inserted into the body but is not fully cleansed and thus sterilized may be prevented from contamination. Therefore, the endoscope device is secure against contamination after it is used.

Since a component that is not sterilized despite cleaning or that is difficult to cleanse can be replaced, the endoscope device can be used for an examinee in a sterilized state. Thus, the examinee is protected against infection. In addition, as an examinee is examined all the time with a sterilized endoscope device which is secure from contamination-caused infection, that is, which does not require additional cleansing, infection caused by germs or virus can be prevented.

While an existing endoscope is not fully sterilized due to a component such as a camera, the endoscope device can be used in a sterilized state simply by replacing the tube unit. Thus an examinee is assured of the safety of the endoscope device. Furthermore, while the existing endoscope device needs a cleaning unit for cleaning, the endoscope device does not require such a cleaning unit.

While the invention has been shown and described with reference to certain embodiments thereof, it will be understood by those skilled in the art that various changes in form and details may be made therein without departing from the spirit and scope of the invention as defined by the appended claims and their equivalents.

What is claimed is:

1. An endoscope device comprising:
a housing connected to a manipulator, the housing configured to be inserted into a human body;
a replaceable tube detachably provided in the housing; and
a guide tube fixedly passing through the housing, to allow the replaceable tube to be inserted thereinto.

2. The endoscope device of claim 1, further comprising a press-fitting unit provided at a front end of the guide tube, to fit a front end of the replaceable tube into the front end of the guide tube by pressing the front end of the replaceable tube.

3. The endoscope device of claim 2, wherein the press-fitting unit comprises:
a distal end having a press-fitting groove on an inner surface of the distal end, provided at the front end of the guide tube; and
a sealing member provided at the press-fitting groove, to seal the front end of the replaceable tube by closely contacting the front end of the replaceable tube with the press-fitting groove.

4. The endoscope device of claim 3, wherein when the replaceable tube is inserted through the front end of the guide tube, the replaceable tube is inserted into the housing along the guide tube, and when the front end of the guide tube is positioned in the press-fitting groove, the sealing member is positioned inside the front end of the guide tube and closely contacts the front end of the guide tube with the distal end, sealing the guide tube.

5. The endoscope device of claim 4, wherein if the sealing member is released from the press-fitting groove and the replaceable tube is pulled from the front end of the guide tube, the replaceable tube is detachable from the guide tube and removable from the housing.

6. An endoscope device comprising:
an inserted part to be inserted into a human body; and
a non-inserted part that is not inserted into the human body,
wherein the inserted part is provided with a tube unit replaceable after one-time use and is connected to the non-inserted part.

7. The endoscope device of claim 6, wherein the inserted part comprises:
a housing connected to the non-inserted part;
a guide tube passing through the housing; and
a replaceable tube detachably inserted into the guide tube.

8. The endoscope device of claim 7, further comprising a press-fitting unit to seal front ends of the guide tube and the replaceable tube by closely contacting the front end of the replaceable tube with the front end of the guide tube.

9. The endoscope device of claim 8, wherein the press-fitting unit comprises:
a distal end having a press-fitting groove on an inner surface of the distal end, provided at the front end of the guide tube; and
a sealing member provided at the press-fitting groove, to seal a gap between the replaceable tube and the guide tube by closely contacting the front end of the replaceable tube with the press-fitting groove.

10. The endoscope device of claim 9, wherein when the replaceable tube is inserted through the front end of the guide tube, the replaceable tube is inserted into the housing along the guide tube, and when the front end of the guide tube is positioned in the press-fitting groove, the sealing member is positioned inside the front end of the guide tube and closely contacts the front end of the guide tube with the distal end, sealing the guide tube.

11. The endoscope device of claim 9, wherein the non-inserted part comprises:
a manipulator connected to the housing;
a suction tube exposed outwardly from the manipulator and connected to a rear end of the replaceable tube; and
a Y branch detachably installed on the manipulator and connecting the suction tube to the replaceable tube.

12. The endoscope device of claim 11, further comprising a fixture to fixedly attach or removed the Y branch to or from an outer surface of the housing.

13. The endoscope device of claim 12, wherein the fixture comprises:
a Y branch fixing surface provided in the housing, exposing the replaceable tube, and engaged with the Y branch so that the Y branch is detachable; and
an engagement portion provided in the Y branch, covering the replaceable tube, and detachably engaged with the Y branch fixing surface.

14. The endoscope device of claim 12, wherein the Y branch is replaced along with the replaceable tube, after the endoscope device is used for an examinee.

15. The endoscope device of claim 14, wherein if the Y branch is detached from the manipulator, the suction tube and the replaceable tube are cut, the sealing member is released from the press-fitting groove, and the replaceable tube is pulled from the front end of the guide tube, the replaceable tube is removed from the housing along the guide tube.

16. The endoscope device of claim 11, wherein the suction tube comprises a suction valve to control suction.

17. The endoscope device of claim 16, wherein the suction valve includes a solenoid valve.
